(19) United States
(12) Plant Patent Application Publication
(10) Pub. No.: US 2017/0099754 P1
(43) Pub. Date: Apr. 6, 2017

(54) Male pistachio variety named 'TEJON'

(50) ;

(71) Applicant: The Regents of the University of California, Oakland, CA (US)

(72) Inventors: Craig E. KALLSEN, Davis, CA (US); Dan E. PARFITT, Davis, CA (US)

(21) Appl. No.: 14/999,157

(22) Filed: Apr. 4, 2016

Related U.S. Application Data
(60) Provisional application No. 62/236,801, filed on Oct. 2, 2015.

(51) Int. Cl. A01H 5/00

(52) U.S. Cl. PLT/152

(57) ABSTRACT

A new male pistachio variety (Pistacia vera L.) designated as ‘Tejon’ is provided. This variety exhibits earlier flowering dates compared to the industry standard male pistachio variety ‘Randy’, and also produces large quantities of relatively durable and viable pollen.

### CROSS-REFERENCE TO RELATED APPLICATION

This application claims the benefit of U.S. Provisional Application No. 62/236,801, filed Oct. 2, 2015, which is incorporated herein by reference in its entirety.

Latin name: Botanical/commercial classification: (Pistacia vera L.), new pistachio variety.

### Varietal denomination: The varietal denomination of the claimed pistachio variety is ‘Tejon’.

### BACKGROUND OF THE INVENTION

An objective of pistachio breeding programs is to develop new varieties that can be harvested at unique times relative to other pistachio varieties. The female pistachio variety ‘Kerman’ is the main later-season pistachio cultivar grown in California and in other parts of the world, but other female pistachio varieties are also grown, such as ‘Golden Hills’. A major problem for pistachio growers that has developed with the rapid increase in pistachio plantings in California, for example, is the availability of harvest equipment/contractors and processing capacity, since most of the existing crop (‘Kerman’) matures at about the same time. ‘Golden Hills’ has become a popular alternative to ‘Kerman’ because it can be harvested before ‘Kerman’, and other early-flowering females pistachio varieties have also been developed (e.g. ‘Gumdrop’). The development of multiple female pistachio varieties that flower in a time series is advantageous because this will allow for increased availability of pistachio harvesting equipment and processing capacity, as well as make available different pistachio varieties that can be harvested in a time series.

Developing a harvest date series is an optimal way to use existing harvest equipment/contractor and processing plant resources. Without staggered harvest dates, the pistachio industry will have to develop significantly increased (e.g. 2×) harvesting capability (which is now provided by custom harvestors) and find investors willing to fund new processing plants (which are only used for a few weeks per year). Failure to develop these facilities or alternatively, a harvest date series, will expose the industry to significant risk from aflatoxin contamination in the crop due to expended pre-processing times.

In order to develop a successful harvest date series, male pistachio varieties whose flowering times sufficiently overlap with that of appropriate female pistachio varieties will need to be available. Female pistachio varieties require a properly synchronized male pollinizer to ensure fruit set, but even the earliest-flowering male pistachio variety being used in the industry (‘Randy’) flowers too late or has poor flowering overlap to pollinate even earlier-flowering female pistachio varieties (e.g. ‘Gumdrop’). Male pistachio varieties that exhibit better flowering synchrony and overlap with early-flowering female pistachio varieties than does ‘Randy’ will aid in developing a successful harvest date series.

Further, to reduce the incidence of insufficient pollen during bloom due to poor synchrony between male and female pistachio trees in low-chill years, the pistachio industry is moving back to the older practice of including earlier-blooming males in orchard designs. For example, growers are commonly adding ‘Randy’ to new ‘Kerman’ plantings, or grafting ‘Randy’ onto ‘Peters’ trees in existing plantings. It would be advantageous to have additional male varieties that can pollinize even earlier than ‘Randy’ to provide additional early pollen in low-chill years.

Thus, there exists a need for improved male pistachio varieties with earlier flowering dates than the present industry standards. The present male pistachio variety ‘Tejon’ described herein is a product of the breeding efforts to produce improved pistachio varieties.

### SUMMARY OF THE INVENTION

The present invention relates to a new and distinct pistachio cultivar (Pistacia vera L.) which has been denominated as ‘Tejon’, and more particularly as a male pistachio variety which exhibits earlier flowering dates compared to the industry standard ‘Randy’ cultivar.

‘Tejon’ produces a large number of flowers and large quantities of durable pollen with a high germination ratio. ‘Tejon’ is a very early flowering male pistachio variety, flowering 6 to 10 days earlier than the male pistachio variety ‘Randy’. The flowering time of ‘Tejon’ also overlaps with the bloom period of the female pistachio variety ‘Gumdrop’. ‘Gumdrop’ flowers much earlier than currently planted female pistachio cultivars, thus ‘Tejon’ should serve the pistachio industry well as a suitable pollinizer variety for ‘Gumdrop’. It is believed that ‘Tejon’ will provide the needed pollination overlap with ‘Gumdrop’ and other early-flowering female pistachio varieties across multiple years and with differing winter chilling seasons. ‘Tejon’ may also serve as a significant pollinizer across diverse climates including, for example, California, New Mexico, Arizona, and other regions of pistachio production, especially those areas that exhibit lower winter chilling. Pistachio growers and/or nursery companies producing budded pistachio cultivars may be consumers of ‘Tejon’, as would growers producing nuts (who would probably obtain budwood or budded trees from a nursery source). ‘Tejon’ may be used as a pollinizer of very early season female pistachio varieties. ‘Tejon’ may also be included as a male pollinizer variety in plantings of female pistachio varieties such as e.g. ‘Golden Hills’ and ‘Lost Hills’ to provide additional early pollen in low-chill years, or to supplement ‘Randy’ pollen.

‘Tejon’ was originally identified as an open-pollinated offspring of female pistachio seedling selection ‘B4-19’, the open-pollination having taken place in a pistachio breeding program test plot near Bakersfield, Calif. during Year 0. The initial seedling was originally designated as selection ‘N-48’. During the open-pollination that produced ‘N-48’, three male pistachio varieties were located near the female selection ‘B4-19’: male varieties ‘Peters’, ‘Randy’, and ‘B6-6’. Since both ‘Peters’ and ‘B6-6’ are very late flowering males (‘B6-6’ is later than ‘Peters’), it is believed that ‘Tejon’ was the result of a cross between the ‘B4-19’ female (a very early flowering selection) and ‘Randy’ (the early flowering male variety at that location). The female selection ‘B4-19’ is a cloned seedling from a cross between a Pistacia vera female (‘2-35’) and an unknown Pistacia vera male, and the male variety ‘Randy’ is the cloned progeny from a cross of the female selection ‘2-35’ and the male selection ‘ES#3’.

The original seedling ‘N-48’ was collected and germinated at an extension office in Bakersfield, Calif. in February of Year 1, and planted and grown in a small test plot at this location in Year 1. ‘N-48’ was later cloned (grafted onto rootstocks) and established in replicated advanced selection trials at a Buttonwillow test plot and a test plot designated as the Jasmine trial, where ‘N-48’ was asexually reproduced when ‘N-48’ budwood was taken and grafted onto pistachio rootstocks to establish this variety in these plots in Year 12. ‘N-48’ is also present as a single tree grafted onto rootstock in the UC pistachio germplasm repository located in Bakersfield, Calif. ‘N-48’ was not an original entry in the Buttonwillow and Jasmine trials. After the first season, when initial flowering was observed at the Buttonwillow trial, several of the female selections in that trial (e.g. ‘Gumdrop’) bloomed earlier (compared to the male variety ‘Randy’ bloom) when grafted on rootstocks than when on their own roots. Because these female selections need a pollinizer with a bloom period that overlaps with their own, ‘N-48’ was grafted into the trial in Year 12. ‘N-48’ was chosen because this selection had a long bloom period and was at full bloom about four days earlier than ‘Randy’. ‘N-48’ also rated a 4 (1=low, 4=high) on a flower density scale, and each inflorescence produced copious pollen with good germination. The precociousness of ‘N-48’, initially observed in the seedling selection trials, was further demonstrated by the observation that it bloomed only two years after it was grafted onto an existing tree in the Buttonwillow trial. ‘N-48’ is at full bloom at least two weeks before ‘Kerman’, with little or no overlap with ‘Kerman’.

Selection ‘N-48’ was chosen as a candidate for release under the variety name ‘Tejon’, and will be useful as a male pollinizer of early-flowering female pistachio varieties. It is believed that use of earlier flowering cultivars will become increasingly common as climate change continues to reduce average chilling periods.

### BRIEF DESCRIPTION OF THE DRAWINGS

FIG. 1 illustrates the mean germination ratio of freshly-collected pollen for ‘Tejon’ and other male pistachio varieties in Year 15. Shown is the analysis of means (MiniTab) with 5% SD, where a value of 1.0=100%.

FIG. 2 illustrates the mean germination ratio of stored pollen for ‘Tejon’ and other male pistachio varieties in Year 15. Shown is the analysis of means (MiniTab) with 5% SD, where a value of 1.0=100%.

FIG. 3 illustrates bloom dates for Years 14-15 for ‘Tejon’ and ‘Gumdrop’ compared to several commercial male and female pistachio varieties. Y-axis shows bloom rating on a scale of 0 to 6 as follows: 0=dormant buds, 1=green tip on flower bud, 2=>(greater than) five open buds on tree, 3=mid bloom, 4=full bloom, 5=late bloom, 6=bloom finished.

FIG. 4 illustrates the mean pollen weight per inflorescence (grams) for ‘Tejon’ and other male pistachio varieties in Year 15. Shown is the analysis of means (MiniTab) with 5% SD.

FIG. 5A-FIG. 5B illustrates a comparison of inflorescence density for ‘Tejon’ (FIG. 5A) and ‘Peters’ (FIG. 5B).

FIG. 6 illustrates the dehiscing inflorescences of ‘Tejon’.

FIG. 7 illustrates the leaves of ‘Tejon’.

### DETAILED BOTANICAL DESCRIPTION

The following is a detailed botanical description of the new male pistachio cultivar designated as ‘Tejon’, including the key differentiating characteristics of this variety and comparisons of certain characteristics of ‘Tejon’ to other pistachio varieties.

Summary of Trials: ‘N-48’ has been evaluated in two randomized, replicated advanced selection trials: the Buttonwillow trial and the Jasmine trial, as described below.

The Buttonwillow trial consists of nine different female selections. Each female selection is in a plot consisting of 6 trees of that selection. Each female plot is replicated three times, once in each of three randomized blocks providing a total of 18 trees of each female selection. Each block is separated by a male row containing replicated males of ‘Peters’, ‘Randy’, and ‘Tejon’. There are two of these replicated male rows within the trial. The trial is surrounded by typical male rows containing ‘Peters’. Flowering/bloom data presented herein for ‘Tejon’ and comparative varieties were obtained from the Buttonwillow trial.

The Jasmine trial contains additional different female and male varieties as compared to the Buttonwillow trial. The Jasmine trial is also randomized and replicated in a similar fashion as the Buttonwillow trial. ‘Tejon’ is demonstrating precociousness in this trial as well, and the ‘Tejon’ trees (budded in Year 12) have caught up to the other male varieties, and are covered with flower buds.

‘Tejon’ has also been evaluated in Bakersfield, Calif. and another trial location near Famoso, Calif. Pollen data presented herein for ‘Tejon’ was obtained from plants at these locations.

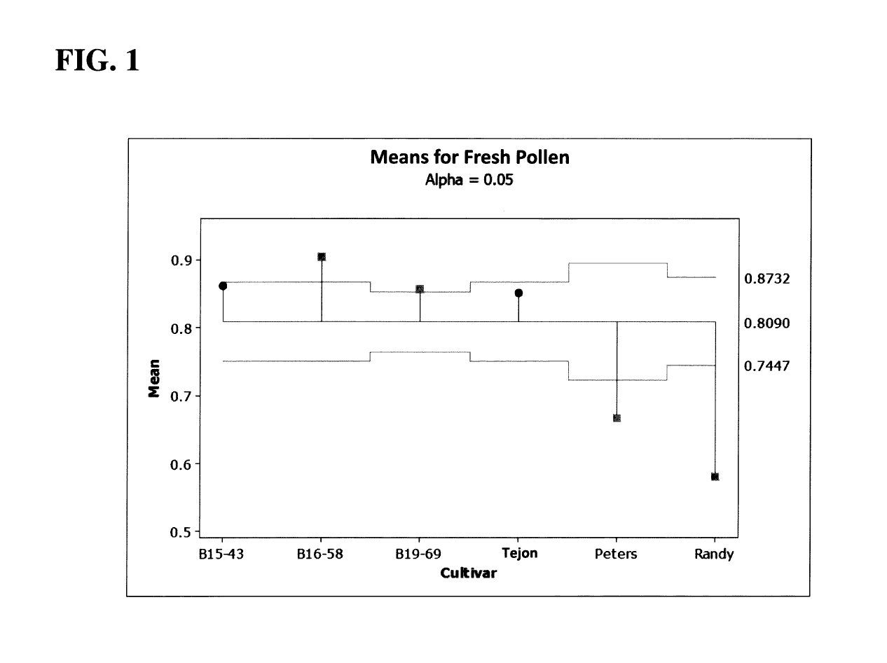

Fresh Pollen Germination: ‘Tejon’ pollen germination at pollen shed was high and significantly higher than ‘Randy’ or ‘Peters’ (Table 1A, Table 1B, FIG. 1). ‘Randy’ has produced large quantities of very viable pollen in prior tests and the current results may reflect differences in flower maturity when ‘Randy’ was collected (e.g. mid bloom vs. later flowering).

TABLE 1A
Least Squares Means for germination of fresh pollen in Year 15,
counted immediately after flowering.

| a) | | |
|---|---|---|
| Cultivar | Mean % | SE Mean % |
| Tejon | 85.09 | 2.334 |
| Peters | 66.67 | 3.301 |
| Randy | 58.09 | 2.557 |
| B15-43 | 86.19 | 2.334 |
| B16-58 | 90.40 | 2.334 |
| B19-69 | 85.66 | 1.906 |

TABLE 1B
GLM ANOVA showing highly significant differences among cultivars.
GLM Analysis of Variance for Germination of Fresh Pollen

| Source | DF | SS | MS | F | P |
|---|---|---|---|---|---|
| Cultivar | 5 | 0.84564 | 0.16913 | 25.87 | 0.000 |
| Error | 64 | 0.41841 | 0.00654 | | |
| Total | 69 | 1.26405 | | | |

S = 0.0808560
R-Sq = 66.90%
R-Sq(adj) = 64.31%

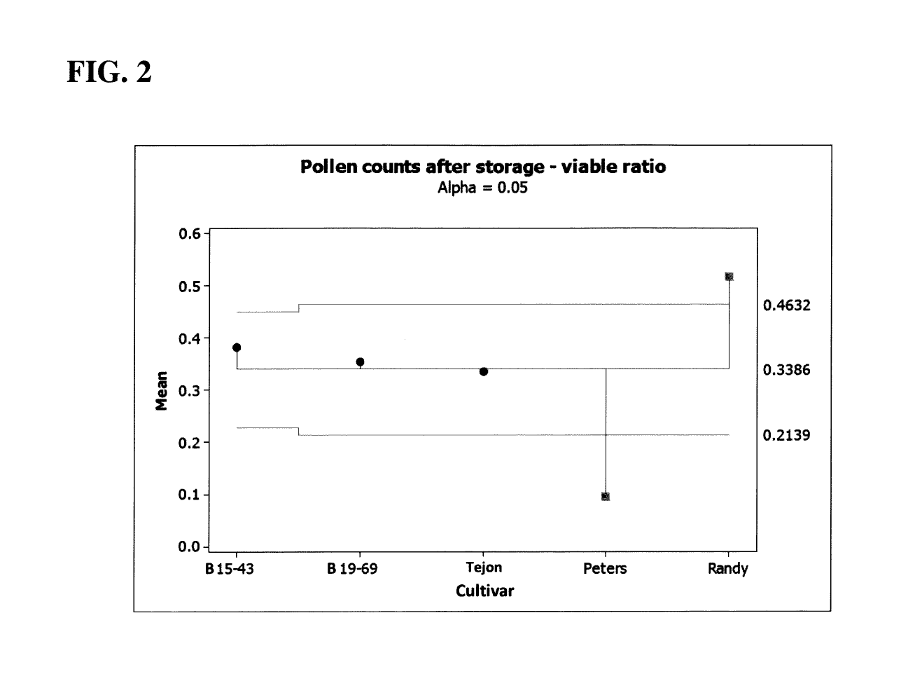

Pollen Viability After Storage: A second set of pollen counts was done after the pollen was stored desiccated at 4°C. for 3 to 4 days (Table 2A, Table 2B, FIG. 2). ‘Randy’ pollen was significantly more durable than other male varieties, and ‘Peters’ pollen was less durable than the other males. ‘Tejon’ was intermediate in pollen germination after 4 days in storage. In prior tests of many males, both ‘Peters’ and ‘Randy’ had better cold-storage durability than most other males evaluated. The overall evaluation of ‘Tejon’ showed that pollen viability was good and sufficient, even in the low-chill Year 15 (Table 3), to provide sufficient pollination for females with which it has a flowering period overlap.

TABLE 2A
Least Squares Means for germination of pollen in Year 15,
counted after 4° C. storage.

| Cultivar | Mean % | SE Mean % |
|---|---|---|
| Tejon | 33.60 | 4.915 |
| Peters | 9.40 | 4.915 |
| Randy | 51.73 | 4.915 |
| B15-43 | 38.20 | 4.487 |
| B19-69 | 35.48 | 4.915 |

TABLE 2B
GLM ANOVA showing highly significant differences among cultivars.
GLM Analysis of Variance for Germination of Stored Pollen

| Source | DF | SS | MS | F | P |
|---|---|---|---|---|---|
| Cultivar | 4 | 0.47137 | 0.11784 | 9.76 | 0.000 |
| Error | 21 | 0.25368 | 0.01208 | | |
| Total | 25 | 0.72504 | | | |

S = 0.109908
R-Sq = 65.01%
R-Sq(adj) = 58.35%

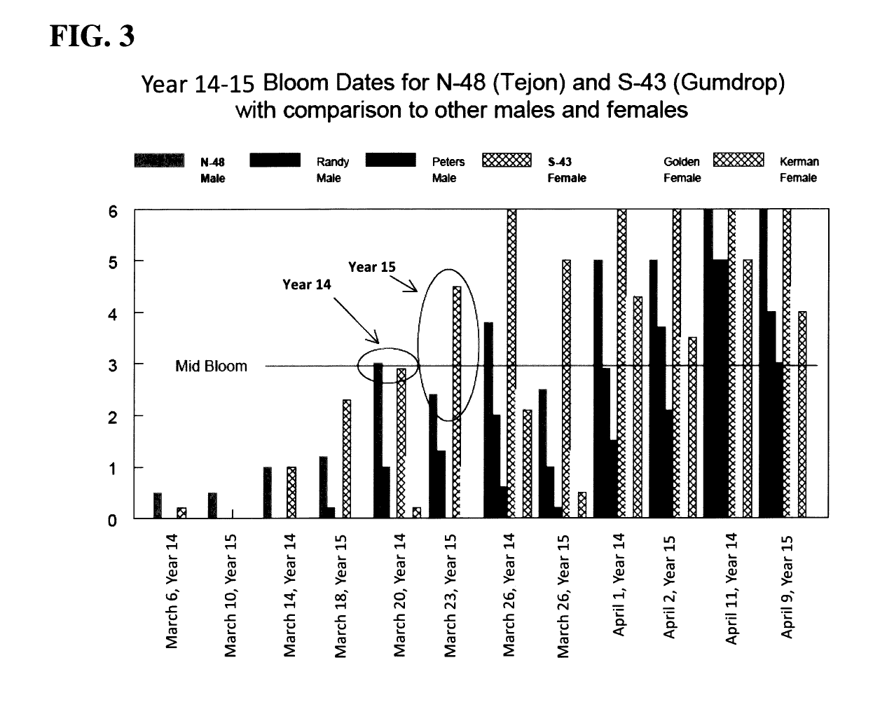

Flowering dates: Flowering was evaluated on the whole tree using a visual scale from 0 to 6, where 0=dormant buds, 1=green tip on flower bud, 2=>(greater than) five open buds on tree, 3=mid bloom, 4=full bloom, 5=late bloom, 6=bloom finished. These scores are averages, since there are differences both within and between trees due to variables such as, for example, the amount of sun exposure, location of the trees in the orchard, location of the orchard, etc. Scores of 3 to 4 provide the most viable pollen for the female trees. Table 3 shows flowering dates for several males (shaded) and their relationship to female cultivars. Years 14 and 15 were low chill years and both ‘Randy’ and ‘Peters’ were delayed relative to ‘Kerman’. Usually, ‘Peters’ is somewhat coincident with ‘Kerman’, although it has a long term reputation for somewhat later flowering relative to ‘Kerman’, especially in low-chill years. In Year 15, ‘Randy’ actually overlapped with both ‘Kerman’ and ‘Golden Hills’, although it was developed as a pollinizer for ‘Golden Hills’ which is usually earlier than ‘Kerman’ (FIG. 3). ‘Tejon’ had a good overlap with ‘Gumdrop’, while no other male variety was early enough to pollinize ‘Gumdrop’.

In the low chill years of Year 14 and Year 15, both ‘Peters’ and ‘Randy’ were six days later than their female counterparts ‘Kerman’ and Golden Hills, respectively. However, ‘Tejon’ and ‘Gumdrop’ remained more closely synchronized. ‘Tejon’ is thus a suitable pollinizer for the early flowering and harvesting female variety ‘Gumdrop’.

TABLE 3
Bloom maturity evaluation scores by date for Years 14 and 15. Note mid-bloom
(3)/full bloom (4) dates overlap.

| | | Evaluation Date: | | | | | |
|---|---|---|---|---|---|---|---|
| Cultivar | | March 6, | March 14, | March 20, | March 26, | April 1, | April 11, |
| Year 14 | | Year 14 | Year 14 | Year 14 | Year 14 | Year 14 | Year 14 |
| Tejon | Male | 0.5 | 1 | 3.0 | 3.8 | 5 | 6 |
| Randy | Male | 0 | 0 | 1.0 | 2 | 2.9 | 5 |
| Peters | Male | 0 | 0 | 0.0 | 0.6 | 1.5 | 4 |
| Gumdrop | Female | 0.2 | 1 | 2.9 | 6 | 6 | 6 |
| Golden Hills | Female | 0 | 0 | 1.2 | 2.5 | 4.3 | 6 |
| Kerman | Female | 0 | 0 | 0.2 | 2.1 | 3 | 5 |
| | | Evaluation Date: | | | | | |
| | | March 10, | March 18, | March 23, | March 26, | April 2, | April 9, |
| Year 15 | | Year 15 | Year 15 | Year 15 | Year 15 | Year 15 | Year 15 |
| Tejon | Male | 0.5 | 1.2 | 2.4 | 2.5 | 5 | 6 |
| Randy | Male | 0 | 0.2 | 1.3 | 1 | 3.7 | 4 |
| Peters | Male | 0 | 0 | 0 | 0.2 | 2.1 | 3 |
| Gumdrop | Female | 0 | 2.3 | 4.5 | 5 | 6 | 6 |
| Golden Hills | Female | 0 | 0 | 1 | 1.3 | 3.5 | 4.5 |
| Kerman | Female | 0 | 0 | 0 | 0.5 | 3.5 | 4 |

Bloom ratings 0 to 6:
0 = dormant buds,
1 = green tip on flower bud,
2 = >5 open buds on tree,
3 = mid bloom,
4 = full bloom,
5 = late bloom,
6 = bloom finished

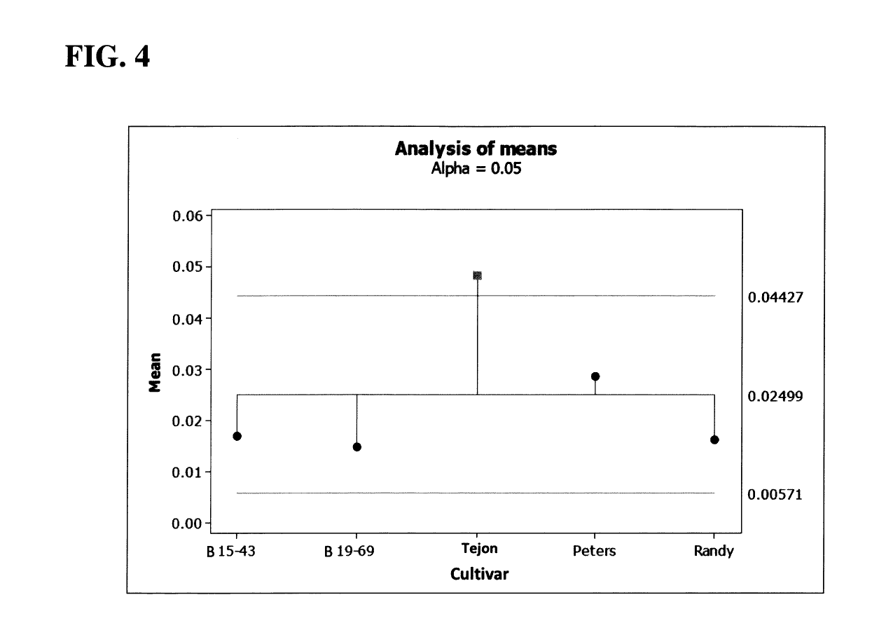

Pollen quantities: During March of Year 15, branches with dehiscing inflorescences were collected and evaluated. Treatments involved taking four to five shoots that were 8-12 inches long with dehiscing inflorescences and placing them on craft paper overnight, followed by pollen collection the following morning. Pollen from three replicates of each treatment were collected and weighed. Treatments were normalized by counting the number of actively dehiscing inflorescences. ANOVA and ANOMA were performed with MiniTab 14. ‘Tejon’ proved, by comparison to other existing male cultivars, to have good quantities of pollen available for pollination (Table 4A, Table 4B, and FIG. 4).

TABLE 4A
Least Squares Means for pollen quantities (grams/inflorescence).

| Accession | Mean | SE Mean |
|---|---|---|
| Tejon | 0.04833 | 0.007042 |
| Peters | 0.02863 | 0.007042 |
| Randy | 0.01627 | 0.007042 |
| B15-43 | 0.01683 | 0.007042 |
| B19-69 | 0.01487 | 0.007042 |

TABLE 4B
GLM ANOVA for pollen quantities per inflorescence.
GLM for pollen quantification

| Source | DF | SS | MS | F | P |
|---|---|---|---|---|---|
| Cultivar | 4 | 0.002410 | 0.000602 | 4.05 | 0.033 |
| Error | 10 | 0.001488 | 0.000149 | | |
| Total | 14 | 0.003898 | | | |

S = 0.01220
R-Sq = 61.83%
R-Sq(adj) = 46.56%

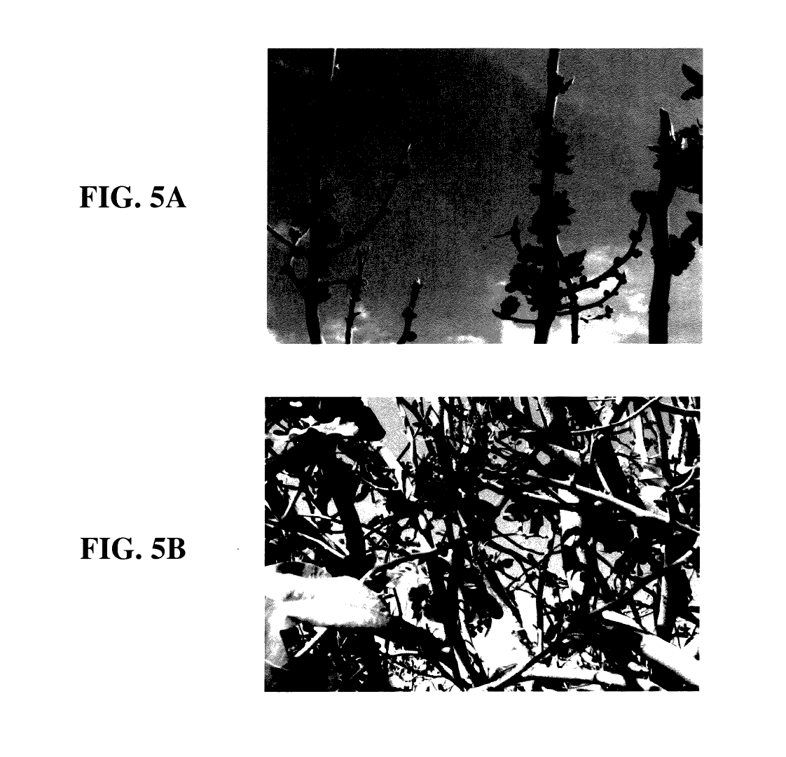

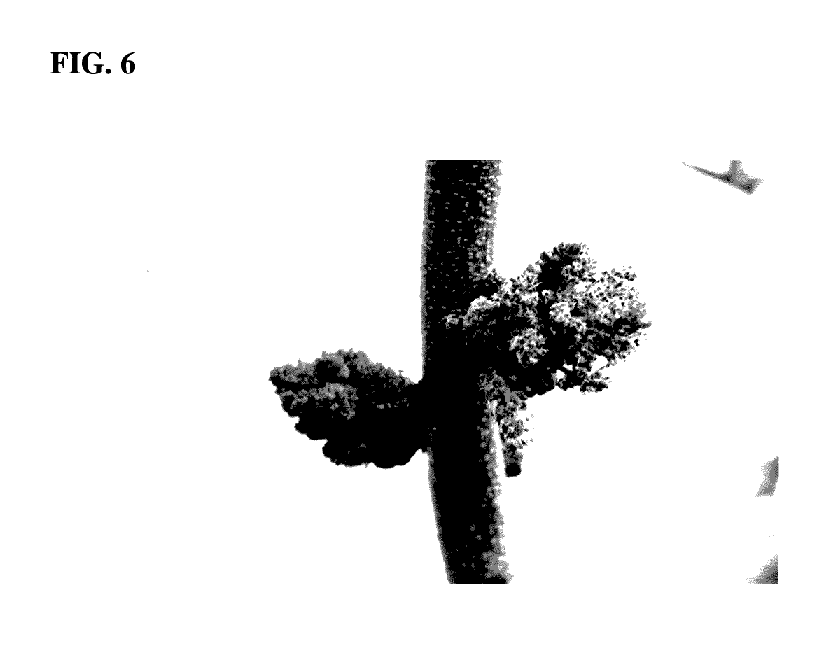

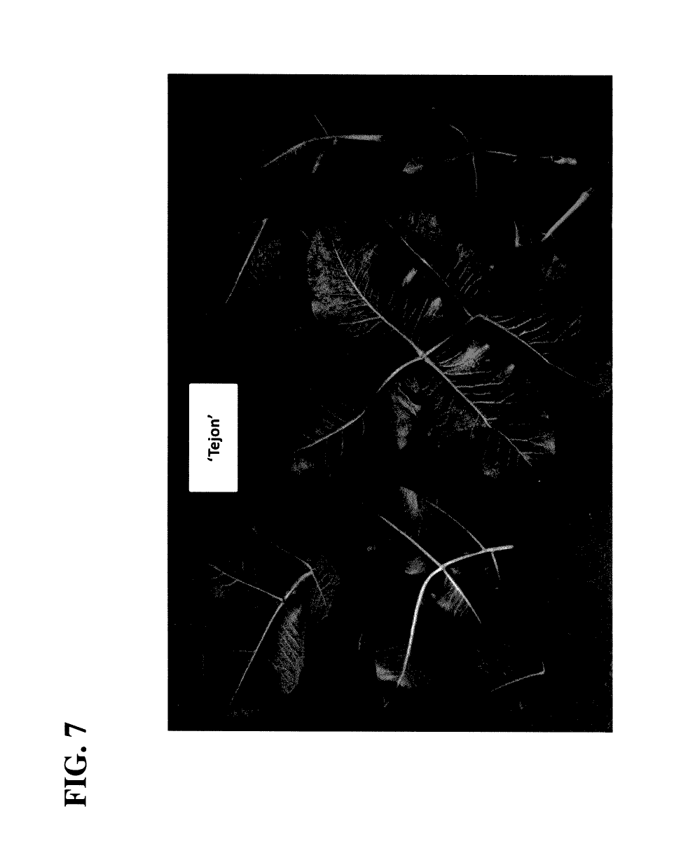

Inflorescence Density: The tree canopies of several male cultivars were photographed to provide an approximate evaluation of the number of inflorescences in the canopy. It is difficult to develop methods that accurately quantify this variable, which is highly dependent on tree size, pruning, and tree health. FIG. 5A and FIG. 5B provide a visual comparison of ‘Tejon’ and ‘Peters’, taken at their respective bloom periods. ‘Peters’ had very scattered bloom in Year 15, with many buds never breaking dormancy. An image of the dehiscing inflorescences of ‘Tejon’ is provided in FIG. 6.
Color of leaves: For leaves, color evaluations were done on ‘Tejon’ and ‘Randy’ (as a comparison) leaves collected during the summer of Year 14. Color descriptions are based on the color standards presented in Royal Horticultural Society; R.H.S. colour chart v. 3 QC495 R8 V.3. The results are presented in Table 5 below. An image of the leaves of ‘Tejon’ is presented in FIG. 7.

TABLE 5
Leaf color evaluation for ‘Tejon’ and ‘Randy’

| | Mature | Mature Leaf- | New | New Leaf- | |
|---|---|---|---|---|---|
| Selection | Leaf-Top | Bottom | Leaf-Top | Bottom | Midrib |
| ‘Randy’ | 136A, | 135B | 137C | 137D | 141D, |
| | 136B | | | | 143D |
| ‘Tejon’ | 136C | 139B | 139C | 139D | 143D |
| (‘N-48’) | | | | | |

What is claimed is:

1. A new and distinct variety of pistachio tree designated ‘Tejon’ as shown and described herein.
